(19) United States
(12) Patent Application Publication
(10) Pub. No.: US 2026/0055153 A1
(43) Pub. Date: Feb. 26, 2026

(54) Development and Use of Novel Interleukin 15 Mutant Polypeptide

(71) Applicant: Suzhou Forlong Biotechnology Co., Ltd., Jiangsu (CN)

(72) Inventors: En Ji, Jiangsu (CN); Chunyu Wang, Jiangsu (CN)

(21) Appl. No.: 18/564,329

(22) PCT Filed: May 23, 2022
(86) PCT No.: PCT/CN2022/094443

§ 371 Date: Nov. 27, 2023

(30) Foreign Application Priority Data
May 28, 2021 (CN) 202110591118.6

(51) Int. Cl. C07K 14/54; A61K 38/00

(52) U.S. Cl. CPC C07K 14/5443; A61K 38/00

(57) ABSTRACT

Provided in the present application are an IL-15 mutant polypeptide, and a preparation method therefor and the use thereof. Further provided are a nucleic acid encoding the IL-15 mutant polypeptide, a vector containing the nucleic acid, and a host cell.

### CROSS REFERENCE TO RELATED APPLICATIONS

The present application is a section 371 of International Application No. PCT/CN2022/094443 filed May 23, 2022, which was published in the Chinse language on Dec. 1, 2022, under International Publication No. WO 2022/247778 A1, which claims the priority of Chinese patent application No. 202110591118.6 entitled “Development and Use of Novel Interleukin 15 Mutant Polypeptide,” filed with the China Patent Office on May 28, 2021, the entire contents of which are incorporated herein by reference.

### REFERENCE TO SEQUENCE LISTING SUBMITTED ELECTRONICALLY

This application contains a sequence listing, which is submitted electronically as an ASCII formatted sequence listing with a file name “069777-1US1 Substitute Sequence Listing” and a creation date of Oct. 3, 2024 and having a size of 19198 bytes. The sequence listing submitted via Patent Center is part of the specification and is herein incorporated by reference in its entirety.

### TECHNICAL FIELD

The present invention belongs to the field of biotechnology, and specifically relates to a novel interleukin 15 mutant polypeptide, a preparation method thereof, and a nucleic acid, a plasmid, and a host cell required for constructing the novel interleukin 15 mutant polypeptide, as well as a corresponding pharmaceutical composition and uses thereof.

### BACKGROUND ART

Interleukin 15 (IL-15) is a soluble cytokine found by Grabstein et al. in 1994 when detecting the supernatant of monkey kidney epidermal cell line CV21/EBNA. It can maintain the proliferation of CTLL-2 cells, and has attracted much attention as it has many similar biological activities with IL-2. Both IL-2 and IL-15 belong to the IL-2 family, and receptors for the IL-2 family members, including IL-2, IL-4, IL-7, IL-9, IL-15 and IL-21, all contain the γc chain. Among them, IL-2 and IL-15 share common IL-2/IL-15Rβ (CD122) and γc (CD132) receptors. The specific receptor for IL-2 is IL-2Rα (CD25), which is mainly expressed on the surface of activated T cells, and IL-2 is the most important cytokine to promote T cell proliferation. The specific receptor for IL-15 is IL15Rα (CD215).

In nature, IL-15 can bind to the IL-15β/γ receptor on the cell surface and activate the downstream STAT5 signaling pathway, which in turn promotes the differentiation and expansion of CD8+ T cells. IL-2 exists mainly in a free state in vivo, and therefore as long as it binds to the cell surface receptor, it can play its role. In contrast, IL-15 and IL-15Rα are mainly expressed on the surface of DC cells and monocytes, and exists rarely in a free state. Therefore, the activation of IL-15 signaling pathways is more likely to activate the downstream signaling pathways to function after cell-to-cell contact. IL-15 signaling is via the heterotrimeric complex of IL-15Rα, IL-15Rβ and γc. The mechanism of action of IL-15 is trans-presentation, that is, one cell presents its cell membrane-bound IL-15/IL-15Rα complex to another target cell expressing IL-15Rβ and γc receptors, which, in fact, is a “cell-to-cell” interaction. Trans-presentation requires that one cell must contact with another cell to function, which is not only required for regulating the body's immune system, but also to a certain extent, restricts the use of the IL-15/IL-15Rα complex for immunotherapy of related diseases.

Therefore, the development of a novel IL-15/IL-15Rα complex using synthetic biology techniques is of great significance. The present inventors have innovatively developed a novel monomeric Fc based on the antibody IgG's Fc in the field of long-acting proteins using synthetic biology (CN109705211B; 202011161007.3), which is introduced into the novel interleukin 15 mutant polypeptide of the present application. Moreover, by using the mechanism of action of trans-presentation of IL-15, the present inventors have innovatively developed a novel interleukin 15 mutant polypeptide with enhanced or reduced binding or biological activities for IL-15Rβ and γc receptors.

### SUMMARY OF THE INVENTION

The main objectives of the present application are to provide a novel IL-15 mutant polypeptide and the use thereof.

In order to achieve the above objectives, the present invention provides the following technical solutions:

A novel IL-15 mutant polypeptide, comprising the amino acid sequence as set forth in SEQ ID NO: 1, wherein:
X1 is selected from any amino acid of A and N;
X2 is selected from any amino acid of S, D and Y;
X3 is selected from any amino acid of D and F;
X4 is selected from any amino acid of D, G and N;
X5 is selected from any amino acid of E and Y;
X6 is selected from any amino acid of I, Q and K;
X7 is selected from any amino acid of N and Y;
X8 is selected from any amino acid of Q, S, F and A;
X9 is selected from any amino acid of M and H; and
X10 is selected from any amino acid of I, A and H; and
the mutant polypeptide has increased or decreased binding activity for IL-15Rβ/γ compared to the naturally occurring IL-15 amino acid sequence.

As a preferred embodiment of the novel IL-15 mutant polypeptide as described above:
the novel IL-15 mutant polypeptide comprises the amino acid sequence as set forth in SEQ ID NO: 1, wherein X1 is A, X2 is S, X3 is D, X4 is D, X5 is E, X6 is I, X7 is N, X8 is Q, X9 is M, and X10 is I;
preferably, the novel IL-15 mutant polypeptide comprises the amino acid sequence as set forth in SEQ ID NO: 1, wherein X1 is N, X2 is selected from any amino acid of D and Y, X3 is D, X4 is D, X5 is E, X6 is I, X7 is N, X8 is Q, X9 is M, and X10 is I;
more preferably, the novel IL-15 mutant polypeptide comprises the amino acid sequence as set forth in SEQ ID NO: 1, wherein X1 is N, X2 is S, X3 is F, X4 is D, X5 is E, X6 is I, X7 is N, X8 is Q, X9 is M, and X10 is I;
further preferably, the novel IL-15 mutant polypeptide comprises the amino acid sequence as set forth in SEQ ID NO: 1, wherein X1 is N, X2 is S, X3 is D, X4 is selected from any amino acid of G and N, X5 is E, X6 is I, X7 is N, X8 is Q, X9 is M, and X10 is I; and
still further preferably, the novel IL-15 mutant polypeptide comprises the amino acid sequence as set forth in SEQ ID NO: 1, wherein X1 is N, X2 is S, X3 is D, X4 is D, X5 is Y, X6 is I, X7 is N, X8 is Q, X9 is M, and X10 is I.

As a preferred embodiment of the novel IL-15 mutant polypeptide as described above: the novel IL-15 mutant polypeptide comprises the amino acid sequence as set forth in SEQ ID NO: 1, wherein X1 is N, X2 is S, X3 is D, X4 is D, X5 is E, X6 is selected from any amino acid of Q and K, X7 is N, X8 is Q, X9 is M, and X10 is I;
preferably, the novel IL-15 mutant polypeptide comprises the amino acid sequence as set forth in SEQ ID NO: 1, wherein X1 is N, X2 is S, X3 is D, X4 is D, X5 is E, X6 is I, X7 is Y, X8 is Q, X9 is M, and X10 is I;
more preferably, the novel IL-15 mutant polypeptide comprises the amino acid sequence as set forth in SEQ ID NO: 1, wherein X1 is N, X2 is S, X3 is D, X4 is D, X5 is E, X6 is I, X7 is Y, X8 is selected from any amino acid of S, F and A, X9 is M, and X10 is I;
further preferably, the novel IL-15 mutant polypeptide comprises the amino acid sequence as set forth in SEQ ID NO: 1, wherein X1 is N, X2 is S, X3 is D, X4 is D, X5 is E, X6 is I, X7 is N, X8 is Q, X9 is H, and X10 is I; and
still further preferably, the novel IL-15 mutant polypeptide comprises the amino acid sequence as set forth in SEQ ID NO: 1, wherein X1 is N, X2 is S, X3 is D, X4 is D, X5 is E, X6 is I, X7 is N, X8 is Q, X9 is M, and X10 is selected from an amino acid of A and H.

SEQ ID NO: 1
(Amino acid sequence of IL-15 mutant)
NWVX1VIX2X3LKKIEDLIQSMHIDATLYTESDVHPSCKVTAMKCFLLEL
QVISLESGDASIHX4TVX5NLIX6LANX7SLSSNGNVTESGCKECEELEE
KNIKEFLQSFVHIVX8X9FX10NTS
SEQ ID NO: 2
(Amino acid sequence of mutant B1-A1)
NWVAVISDLKKIEDLIQSMHIDATLYTESDVHPSCKVTAMKCFLLELQVI
SLESGDASIHDTVENLIILANNSLSSNGNVTESGCKECEELEEKNIKEFL
QSFVHIVQMFINTS
SEQ ID NO: 3
(Amino acid sequence of mutant B1-H4)
NWVNVIDDLKKIEDLIQSMHIDATLYTESDVHPSCKVTAMKCFLLELQVI
SLESGDASIHDTVENLIILANNSLSSNGNVTESGCKECEELEEKNIKEFL
QSFVHIVQMFINTS
SEQ ID NO: 4
(Amino acid sequence of mutant B1-H5)
NWVNVIYDLKKIEDLIQSMHIDATLYTESDVHPSCKVTAMKCFLLELQVI
SLESGDASIHDTVENLIILANNSLSSNGNVTESGCKECEELEEKNIKEFL
QSFVHIVQMFINTS
SEQ ID NO: 5
(Amino acid sequence of mutant B2-C1)
NWVNVISFLKKIEDLIQSMHIDATLYTESDVHPSCKVTAMKCFLLELQVI
SLESGDASIHDTVENLIILANNSLSSNGNVTESGCKECEELEEKNIKEFL
QSFVHIVQMFINTS
SEQ ID NO: 6
(Amino acid sequence of mutant B2-G1)
NWVNVISDLKKIEDLIQSMHIDATLYTESDVHPSCKVTAMKCFLLELQVI
SLESGDASIHGTVENLIILANNSLSSNGNVTESGCKECEELEEKNIKEFL
QSFVHIVQMFINTS
SEQ ID NO: 7
(Amino acid sequence of mutant B2-H1)
NWVNVISDLKKIEDLIQSMHIDATLYTESDVHPSCKVTAMKCFLLELQVI
SLESGDASIHNTVENLIILANNSLSSNGNVTESGCKECEELEEKNIKEFL
QSFVHIVQMFINTS
SEQ ID NO: 8
(Amino acid sequence of mutant B3-A1)
NWVNVISDLKKIEDLIQSMHIDATLYTESDVHPSCKVTAMKCFLLELQVI
SLESGDASIHDTVYNLIILANNSLSSNGNVTESGCKECEELEEKNIKEFL
QSFVHIVQMFINTS
SEQ ID NO: 9
(Amino acid sequence of mutant B4-B1)
NWVNVISDLKKIEDLIQSMHIDATLYTESDVHPSCKVTAMKCFLLELQVI
SLESGDASIHDTVENLIQLANNSLSSNGNVTESGCKECEELEEKNIKEFL
QSFVHIVQMFINTS
SEQ ID NO: 10
(Amino acid sequence of mutant B4-B6)
NWVNVISDLKKIEDLIQSMHIDATLYTESDVHPSCKVTAMKCFLLELQVI
SLESGDASIHDTVENLIKLANNSLSSNGNVTESGCKECEELEEKNIKEFL
QSFVHIVQMFINTS
SEQ ID NO: 11
(Amino acid sequence of mutant B4-E1)
NWVNVISDLKKIEDLIQSMHIDATLYTESDVHPSCKVTAMKCFLLELQVI
SLESGDASIHDTVENLIILANYSLSSNGNVTESGCKECEELEEKNIKEFL
QSFVHIVQMFINTS
SEQ ID NO: 12
(Amino acid sequence of mutant R2-F7)
NWVNVISDLKKIEDLIQSMHIDATLYTESDVHPSCKVTAMKCFLLELQVI
SLESGDASIHDTVENLIILANNSLSSNGNVTESGCKECEELEEKNIKEFL
QSFVHIVSMFINTS
SEQ ID NO: 13
(Amino acid sequence of mutant R2-G7)
NWVNVISDLKKIEDLIQSMHIDATLYTESDVHPSCKVTAMKCFLLELQVI
SLESGDASIHDTVENLIILANNSLSSNGNVTESGCKECEELEEKNIKEFL
QSFVHIVFMFINTS
SEQ ID NO: 14
(Amino acid sequence of mutant R2-B9)
NWVNVISDLKKIEDLIQSMHIDATLYTESDVHPSCKVTAMKCFLLELQVI
SLESGDASIHDTVENLIILANNSLSSNGNVTESGCKECEELEEKNIKEFL
QSFVHIVAMFINTS
SEQ ID NO: 15
(Amino acid sequence of mutant R3-D1)
NWVNVISDLKKIEDLIQSMHIDATLYTESDVHPSCKVTAMKCFLLELQVI
SLESGDASIHDTVENLIILANNSLSSNGNVTESGCKECEELEEKNIKEFL
QSFVHIVQHFINTS
SEQ ID NO: 16
(Amino acid sequence of mutant R4-E5)
NWVNVISDLKKIEDLIQSMHIDATLYTESDVHPSCKVTAMKCFLLELQVI
SLESGDASIHDTVENLIILANNSLSSNGNVTESGCKECEELEEKNIKEFL
QSFVHIVQMFANTS
SEQ ID NO: 17
(Amino acid sequence of mutant R4-G8)
NWVNVISDLKKIEDLIQSMHIDATLYTESDVHPSCKVTAMKCFLLELQVI
SLESGDASIHDTVENLIILANNSLSSNGNVTESGCKECEELEEKNIKEFL
QSFVHIVQMFHNTS.

In a second aspect of the present invention, there is provided a nucleic acid molecule encoding the novel IL-15 mutant polypeptide as described above.

In a third aspect of the present invention, there is provided a plasmid, comprising the nucleic acid molecule as described above.

In a fourth aspect of the present invention, there is provided a host cell, comprising the plasmid as described above.

In a fifth aspect of the present invention, there is provided a fusion, formed by linking a fusion partner to the N-terminal and/or C-terminal of the novel IL-15 mutant polypeptide as described above; wherein
the fusion partner is at least one of a heterologous protein, an adhesion molecule, a nucleic acid molecule, a small molecule compound, a toxin, an immune cell, and a detectable marker;
the heterologous protein is an antibody, an antigen, a cytokine, a soluble receptor domain, a ligand, an enzyme, a peptide, or a protein domain, and the antigen is derived from an adenovirus-associated virus; and
the immune cell is a chimeric antigen receptor T cell.

In a sixth aspect of the present invention, there is provided a pharmaceutical composition, comprising a prophylactically or therapeutically effective dose of the novel IL-15 mutant polypeptide as described above, or the fusion as described above, and a pharmaceutically acceptable carrier.

In a seventh aspect of the present invention, there is provided use of the IL-15 mutant polypeptide as described above or the pharmaceutical composition as described above in the preparation of a medicament for the treatment of a biological material.

In an eighth aspect of the present invention, there is provided a method for treating an autoimmune disease, an inflammatory disease, a neurodegenerative disease, a cancer or a pathogen infection, comprising administering to a subject a therapeutically effective amount of the novel IL-15 mutant polypeptide as described above, the nucleic acid molecule as described above, the plasmid as described above, or the fusion as described above.

In a ninth aspect of the present invention, there is provided a detection kit, comprising the novel IL-15 mutant polypeptide as described above, the nucleic acid molecule as described above, the plasmid as described above, or the fusion as described above; wherein
the detection kit as described above is used for detecting a pathogen and a tumor cell.

Compared with the prior art, the present invention has the following beneficial effects: The present invention innovatively develops a novel interleukin 15 mutant polypeptide with enhanced binding or biological activities for IL-15Rγ and γc receptors, provides a nucleic acid sequence encoding the novel interleukin 15 mutant polypeptide and the amino acid sequence thereof, and in some embodiments, discloses a vector comprising the nucleic acid sequence and a host cell comprising the vector.

The present invention also discloses a pharmaceutical composition comprising the novel interleukin 15 mutant polypeptide. The antibody and composition disclosed herein can be used in the diagnosis and treatment of a disease. In embodiments, the novel interleukin 15 mutant polypeptide, the nucleic acid, the plasmid, and the vector are used for diagnosing or treating a related disease such as an autoimmune disease or a cancer.

### BRIEF DESCRIPTION OF THE DRAWINGS

FIG. 1a shows the reduced SDS-PAGE analysis and Coomassie blue staining pattern of novel interleukin 15 mutant polypeptides (WT-IL15, B1-A1, B1-H4, B1-H5, B2-C1, B2-G1, B2-H1, B3-A1, B4-B1 and B4-B6).

FIG. 1b shows the reduced SDS-PAGE analysis and Coomassie blue staining pattern of novel interleukin 15 mutant polypeptides (B4-E1, R2-F7, R2-G7, R2-B9, R3-D1, R4-E5 and R4-G8).

FIG. 2a shows the affinity of novel interleukin 15 mutant polypeptides (WT-IL15, B2-G1, B2-H1 and B3-A1) for IL-15Rβ as detected by Bio-Layer Interferometry (BLI).

FIG. 2b shows the affinity of novel interleukin 15 mutant polypeptides (B4-B1, B4-B6, B4-E1 and R1-E5) for IL-15Rβ as detected by Bio-Layer Interferometry (BLI).

FIG. 3 shows the biological activity of novel interleukin 15 mutant polypeptides as evaluated by a proliferation assay on CTLL-2.

### DETAILED DESCRIPTION OF EMBODIMENTS

In order to enable persons skilled in the art to better understand the solutions of the present application, the following clearly and completely describes the technical solutions in the embodiments of the present application with reference to embodiments. Apparently, the described embodiments are only a part of the embodiments of the present application, rather than all of the embodiments. Based on the embodiments of the present application, all other embodiments obtained by those of ordinary skill in the art without making creative efforts shall fall within the scope of protection of the present application.

In order that the present invention may be more thoroughly understood, some definitions are set forth below. Said definitions are intended to include grammatical equivalents.

As used herein, “N-terminal,” also referred to as amino-terminus, NH₂-terminus, N-terminus or amine-terminus, is the start of a protein or polypeptide, and refers to the free amine group (—NH₂) at the terminus of a polypeptide. “C-terminal,” also known as carboxy-terminus, carboxyl-terminus, C-terminal tail, C-terminus or COOH-terminus, is the terminus of a protein or polypeptide, terminated by a free carboxy (—COOH) terminus.

As used herein, “amino acid” means one of the 20 naturally occurring amino acids or any non-natural analog thereof, which may be located at a specified position.

By “protein” herein is meant at least two covalently linked amino acids, which includes proteins, polypeptides, oligopeptides and peptides. Proteins can be composed of naturally occurring amino acids and peptide bonds, or of synthetic peptide mimetic structures, i.e., “analogs”.

As used herein, “nucleic acid” means a polymer composed of nucleotide units (ribonucleotides, deoxyribonucleotides, related naturally occurring structural variants, and synthetic non-naturally occurring analogs thereof) linked via phosphodiester bonds. Thus, the term includes polymers of nucleotides, in which the nucleotides and the linkages therebetween include non-naturally occurring synthetic analogs, such as, but not limited to, phosphorothioates, phosphoramidates, methylphosphonates, chiral methylphosphonates, 2′-O-methyl ribonucleotides, peptide nucleic acids (PNAs), and the like. For example, these polynucleotides can be synthesized using an automated DNA synthesizer. The term “oligonucleotide” generally refers to a short polynucleotide, typically no greater than about 50 nucleotides. It will be understood that when a nucleotide sequence is represented by a DNA sequence (i.e., A, T, G and C), this also includes RNA sequences in which “T” is replaced by “U” (i.e., A, U, G and C).

The regular notations are used herein to describe nucleotide sequences: the left-hand end of a single-stranded nucleotide sequence is referred to as the 5′ end; and the left-hand direction of a double-stranded nucleotide sequence is referred to as the 5′ direction. The direction in which nucleotides are added from 5′ to 3′ to the nascent RNA transcript is referred to as the direction of transcription. The DNA strand that has the same sequence as the mRNA is referred to as the coding strand.

As used herein, “encoding” means the intrinsic property of a particular nucleotide sequence in a polynucleotide, such as a gene, a cDNA or an mRNA, to be used as a template for the synthesis of other polymers and macromolecules in a biological process with a defined nucleotide sequence or a defined amino acid sequence, and biological properties resulting therefrom. Thus, a gene encodes a protein if transcription and translation of the mRNA produced by the gene produces the protein in a cell or other biological systems. The coding strand (whose nucleotide sequence is identical to the mRNA sequence and is usually provided in the sequence listing) and the non-coding strand (which is used as a transcription template of a gene or a cDNA) may be said to encode a protein, or other products of the gene or the cDNA. Unless otherwise specified, “a nucleotide sequence encoding an amino acid sequence” includes all nucleotide sequences that are degenerate versions of each other and that encode the same amino acid sequence. Nucleotide sequences that encode proteins and RNA may include introns.

As used herein, “plasmid” means a plasmid artificially constructed on the basis of a natural plasmid for adaptation to laboratory operations, through which a nucleic acid molecule can be introduced into a host cell, thereby producing a transformed host cell. A vector may include a nucleic acid sequence that allows it to replicate in a host cell, such as an origin of replication, and may also include one or more selectable marker genes and other genetic elements known in the art.

As used herein, “host cell”, also referred to as recipient cell, refers to a host cell that receives a foreign gene in transformation and transduction (infection).

As used herein, “pharmaceutically acceptable carrier” means a regular pharmaceutically acceptable carrier. Remington's Pharmaceutical Sciences, E. W. Martin, Mack Publishing Co., Easton, Pa., 15th ed. (1975) describes compositions and formulations suitable for drug delivery of one or more therapeutic compounds or molecules, such as one or more antibodies, as well as additional agents.

As used herein, “preventing” a disease refers to inhibiting the full development of the disease. “Treatment” refers to a therapeutic intervention that ameliorates a sign or symptom of a disease or pathological condition after it has begun to develop.

By “prophylactically/therapeutically effective dose” herein is meant an amount of a particular agent sufficient to achieve the desired effects in a subject treated with the agent. The precise dosage will depend on the purpose of the treatment and can be determined by those skilled in the art using well known techniques. The dosage may be in the range of 0.01-100 mg/kg body weight or more, for example 0.1, 1, 10 or 50 mg/kg body weight, preferably 1-10 mg/kg. As is well known in the art, adjustments may be necessary for antibody or Fc fusion degradation, the rate of systemic or local drug delivery and new protease synthesis, as well as for age, body weight, general health, sex, diet, timing of administration, drug interactions, and severity of the condition, and can be determined by those skilled in the art using regular experimental methods.

By “autoimmune disease” herein is meant a disease in which the immune system generates an immune response (e.g., a B-cell or T-cell response) against a subset of antigens (i.e., self-antigens) of a normal host, with subsequent damage to the tissues. Self-antigens may be derived from host cells, or may be derived from commensal organisms, such as microorganisms that typically colonize mucosal surfaces (referred to as commensals). Autoimmune diseases affecting mammals include, but are not limited to, rheumatoid arthritis, juvenile oligoarthritis, collagen-induced arthritis, adjuvant-induced arthritis, Sjogren's syndrome, multiple sclerosis, experimental autoimmune encephalomyelitis, inflammatory bowel disease (e.g., Crohn's disease, and ulcerative colitis), autoimmune gastric atrophy, pemphigus vulgaris, psoriasis, vitiligo, type 1 diabetes, non-obese diabetes, myasthenia gravis, Graves disease, hashimoto's thyroiditis, sclerosing cholangitis, sclerosing salivary adenitis, systemic lupus erythematosus, autoimmune thrombocytopenic purpura, Goodpasture syndrome, Addison's disease, systemic sclerosis, polymyositis, dermatomyositis, autoimmune hemolytic anemia, pernicious anemia, and the like.

Preferably, the novel interleukin 15 mutant polypeptide of the present invention can be used to treat a cancer. The cancers as described in the present invention include, but are not limited to, lymphomas, blastomas, sarcomas (including liposarcomas), neuroendocrine tumors, mesotheliomas, schwannomas, meningiomas, adenomas, melanomas, and non-leukemic leukemias or lymphoid malignancies. More specific examples of the cancers as described above include squamous cell carcinomas (e.g., squamous epithelial cell carcinoma), lung cancer, small cell lung cancer, non-small cell lung cancer, lung adenocarcinoma and lung squamous cell carcinoma, peritoneal cancer, hepatocellular carcinoma, gastric cancer, gastrointestinal cancer, pancreatic cancer, malignant glioma, cervical cancer, ovarian cancer, liver cancer, bladder cancer, hepatoma, breast cancer, colon cancer, rectal cancer, colorectal cancer, endometrial or uterine cancer, salivary gland cancer, renal cancer, prostate cancer, vulva cancer, thyroid cancer, liver cancer, anal cancer, penis cancer, carcinoma of testis, esophageal cancer, bile duct tumor, head and neck cancer, bone marrow stromal tumor, osteoclastoma, multiple myeloma, osteolytic cancers (osteolytic bone cancers), central nervous system tumor, brain tumors (glioma, neuroblastoma, astrocytoma, medulloblastoma, ependymoma and retinoblastoma), nasopharyngeal carcinoma, basal cell carcinoma, cholangiocarcinoma, Kaposi's sarcoma, primary liver or endometrial carcinoma, and vascular tumors (angiosarcoma and hemagiopericytoma).

The present invention discloses a novel IL-15 mutant polypeptide, comprising the amino acid sequence as set forth in SEQ ID NO: 1, wherein:
X1 is selected from any amino acid of A and N;
X2 is selected from any amino acid of S, D and Y;
X3 is selected from any amino acid of D and F;
X4 is selected from any amino acid of D, G and N;
X5 is selected from any amino acid of E and Y;
X6 is selected from any amino acid of I, Q and K;
X7 is selected from any amino acid of N and Y;
X8 is selected from any amino acid of Q, S, F and A;
X9 is selected from any amino acid of M and H; and
X10 is selected from any amino acid of I, A and H; and
the mutant polypeptide has increased or decreased binding activity for IL-15Rβ/γ compared to the naturally occurring IL-15 amino acid sequence.

The present invention also discloses a nucleic acid molecule encoding the novel IL-15 mutant polypeptide as described above.

The present invention also discloses a plasmid, comprising the nucleic acid molecule as described above.

The present invention also discloses a host cell, comprising the plasmid as described above.

The present invention also discloses a fusion, formed by linking a fusion partner to the N-terminal and/or C-terminal of the novel IL-15 mutant polypeptide as described above; wherein
the fusion partner is at least one of a heterologous protein, an adhesion molecule, a nucleic acid molecule, a small molecule compound, a toxin, an immune cell, and a detectable marker;
the heterologous protein is an antibody, an antigen, a cytokine, a soluble receptor domain, a ligand, an enzyme, a peptide, or a protein domain, and the antigen is derived from an adenovirus-associated virus; and
the immune cell is a chimeric antigen receptor T cell.

The present invention also discloses a pharmaceutical composition, comprising a prophylactically or therapeutically effective dose of the novel IL-15 mutant polypeptide as described above, or the fusion as described above, and a pharmaceutically acceptable carrier.

The present invention also discloses use of the novel IL-15 mutant polypeptide as described above or the pharmaceutical composition as described above in the preparation of a medicament for the treatment of a biological material.

The present invention also discloses a method for treating an autoimmune disease, an inflammatory disease, a neurodegenerative disease, a cancer or a pathogen infection, comprising administering to a subject a therapeutically effective amount of the novel IL-15 mutant polypeptide as described above, the nucleic acid molecule as described above, the plasmid as described above, or the fusion as described above.

The present invention also discloses a detection kit, comprising the novel IL-15 mutant polypeptide as described above, the nucleic acid molecule as described above, the plasmid as described above, or the fusion as described above; wherein the detection kit as described above is used for detecting a pathogen and a tumor cell.

Standard recombinant DNA techniques and molecular cloning techniques used in the Examples are well known in the art (Ausubel, F. M. et al., Current Protocols in Molecular Biology, published by Greene Publishing Assoc. and Wiley-Interscience), and materials and methods suitable for microbial growth are well known in the art. The main chemical and biological reagents were purchased from KAPA Biosystems, New England Biolabs, TransGen Biotech, Thermo Fisher Scientific, OMEGA bio-tek, etc.

The present invention will be described in detail below with reference to specific examples.

### Example 1: Design and Construction of Novel Interleukin 15 Mutant Library

According to the crystalline complex structure (PDB ID: 4GS7) of IL-15 and its receptors α, β and γ, the three receptors bind to three different positions of IL-15, respectively. Therefore, when the interface residues of IL-15 and its receptors β and γ are modified, the binding of IL-15 to receptor a will not be affected.

Therefore, we used the crystal structure to determine the key sites for the interaction of IL-15 with its receptor β to be N4, S7, D8, D61, E64, N65, 168 and N72, and the key sites for the interaction of IL-15 with its receptor γ to be D30, H32, Q108, M109, I111 and N112.

After selecting the key sites of action, we designed and used degenerate amino acid primers to introduce random site-directed mutations at the key amino acid sites, and constructed them into the phage expression vector pComb3x. We innovatively used monoclonal protein capture ELISA, in which IL-15Rβ and γc receptors were used as capture antigens, with which the selected monoclones mutated at key sites were reacted to preliminarily determine the affinity of these novel IL-15 mutants for receptor β and γ, and the wild-type IL-15 was used as control.

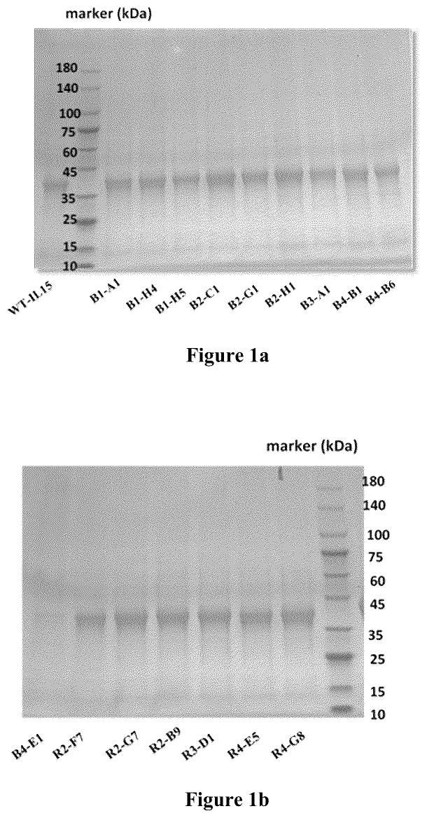

The positive cloned genes were cloned into the eukaryotic expression vector PTT, the successfully constructed plasmids were extracted and transiently co-transformed into Expi293 cells for expression, and purified by protein G resin (YING Tianlei et al., JOURNAL OF BIOLOGICAL CHEMISTRY, 2012, 287(23): 19399-19408). The purity of the purified proteins was verified by SDS-PAGE gel, and the results were shown in FIG. 1.

### Example 2: Detection of Binding Ability of Novel IL-15 Mutant Polypeptides to IL-15Rβ by Bio-Layer Interferometry (BLI)

The Anti-human IgG Fc Capture (AHC) probe (purchased from GE) was used to detect the respective binding ability of the novel interleukin 15 mutant polypeptides to IL-15Rβ.

The expression and preparation of the novel interleukin 15 mutant polypeptides in a soluble form were substantially carried out according to the literature (YING Tianlei et al., JOURNAL OF BIOLOGICAL CHEMISTRY, 2012, 287(23): 19399-19408). During the detection procedure, the novel interleukin 15 mutant polypeptides were diluted at a ratio of 1:3 (the initial concentration for the detection of the affinity for IL-15Rβ was 1000 nM, which was serially diluted to 12.35 nM).

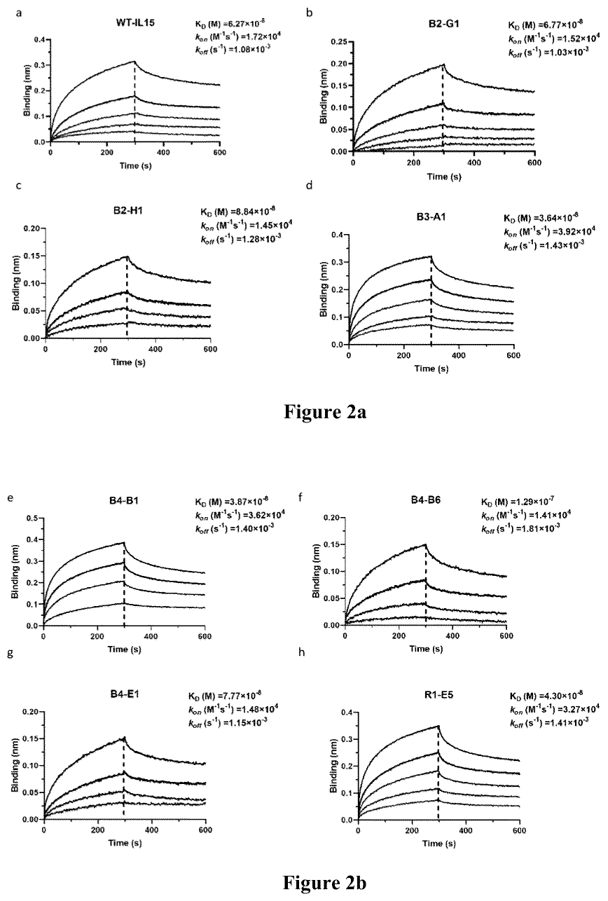

As shown in the results of FIG. 2, the novel interleukin 15 mutant polypeptides had increased or decreased binding to IL-15Rβ due to mutations at the key amino acid sites for binding to IL-15Rβ.

### Example 3: Evaluation of Biological Activity of Novel IL-15 Mutant Polypeptides in Cell Line CTLL-2

To evaluate the biological activity of the novel IL-15 mutant polypeptides, a proliferation assay on cell line CTLL-2 was used. Biological activity was measured by the stimulation of the proliferation of these cells using mitochondrial staining with 3-(4,5-dimethylthiazol-2-yl)-2,5-diphenyltetrazolium bromide (MTT) (Mossman T. J. Immunol. Methods. 1983, 65(1-2): 55-63) according to the procedure described below.

Serial dilutions (starting from a concentration of 4 μg/mL) were performed on B1-A1, B1-H4, B1-H5, B2-C1, B2-G1, B2-H1, B3-A1, B4-B1, B4-B6, B4-E1, R2-F7, R2-G7, R2-B9, R3-D1, R4-E5, R4-G8, WT-15 and sFc in 50 μL of RPMI medium supplemented with 10% fetal bovine serum (FBS) and 50 μg/mL gentamicin in a 96-well culture plate (Costar, USA). CTLL-2 cells were pre-washed 5 times with RPMI medium and added to the plate at 5×10³ cells/well in a volume of 50 μL.

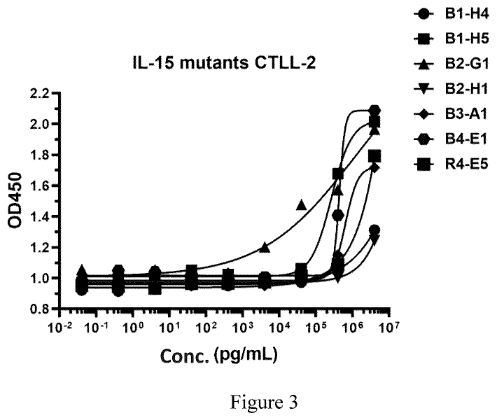

The plate was incubated for 72 hours at 5% CO2 and 98% relative humidity at 37° C. Cell viability was determined by staining with MTT. sFc was not biologically active, because it did not induce the proliferation of CTLL-2 cells, unlike B1-H4, B1-H5, B2-G1, B2-H1, B3-A1, B4-E1 and R4-E5, which stimulated proliferation in a dose-dependent manner, as shown in the results of FIG. 3.

The above description of the disclosed embodiments enables those skilled in the art to implement or use the present invention. The above description is only a preferred implementation of the present invention, and it should be pointed out that for those skilled in the art, several improvements and supplements can be also made without departing from the principles of the present invention, which should also be regarded to fall within the scope of protection of the present invention. Various modifications to these embodiments will be readily apparent to those skilled in the art, and the generic principles defined herein may be implemented in other embodiments without departing from the spirit or scope of the present invention. Thus, the present invention will not be limited to the embodiments shown herein, but is to conform to the widest range consistent with the principles and novel features disclosed herein.

1. An interleukin-15 (IL-15) mutant polypeptide, comprising the amino acid sequence as set forth in SEQ ID NO: 1, wherein:
X1 is selected from any amino acid of A and N;
X2 is selected from any amino acid of S, D and Y;
X3 is selected from any amino acid of D and F;
X4 is selected from any amino acid of D, G and N;
X5 is selected from any amino acid of E and Y;
X6 is selected from any amino acid of I, Q and K;
X7 is selected from any amino acid of N and Y;
X8 is selected from any amino acid of Q, S, F and A;
X9 is selected from any amino acid of M and H; and
X10 is selected from any amino acid of I, A and H;
wherein the mutant polypeptide has increased or decreased binding activity for IL-15Rβ/γ compared to the naturally occurring IL-15 amino acid sequence.

2. The IL-15 mutant polypeptide according to claim 1, wherein the IL-15 mutant polypeptide comprises the amino acid sequence as set forth in SEQ ID NO: 1, wherein X1 is A, X2 is S, X3 is D, X4 is D, X5 is E, X6 is I, X7 is N, X8 is Q, X9 is M, and X10 is I.

3. The IL-15 mutant polypeptide according to claim 1, wherein the IL-15 mutant polypeptide comprises the amino acid sequence as set forth in SEQ TD NO: 1, wherein X1 is N, X2 is S, X3 is D, X4 is D, X5 is E, X6 is selected from any amino acid of Q and K, X7 is N, X8 is Q, X9 is M, and X10 is I.

4. A nucleic acid molecule, encoding the IL-15 mutant polypeptide according to claim 1.

5. A plasmid, comprising the nucleic acid molecule according to claim 4.

6. A host cell, comprising the plasmid according to claim 5.

7. A fusion, formed by linking a fusion partner to the N-terminal and/or C-terminal of the IL-15 mutant polypeptide according to claim 1; wherein
the fusion partner is at least one of a heterologous protein, an adhesion molecule, a nucleic acid molecule, a small molecule compound, a toxin, an immune cell, and a detectable marker;
and wherein the heterologous protein is an antibody, an antigen, a cytokine, a soluble receptor domain, a ligand, an enzyme, a peptide, or a protein domain, and the antigen is derived from an adenovirus-associated virus; and
the immune cell is a chimeric antigen receptor T cell.

8. A pharmaceutical composition, comprising a prophylactically or therapeutically effective dose of the IL-15 mutant polypeptide according to claim 1, and a pharmaceutically acceptable carrier.

9. (canceled)

10. A method for treating an autoimmune disease, an inflammatory disease, a neurodegenerative disease, a cancer or a pathogen infection, comprising administering to a subject a therapeutically effective amount of the IL-15 mutant polypeptide according to claim 1.

11. A detection kit, comprising the IL-15 mutant polypeptide according to claim 1; wherein the detection kit is used for detecting a pathogen or a tumor cell.

12. The IL-15 mutant polypeptide according to claim 1, wherein the IL-15 mutant polypeptide comprises the amino acid sequence as set forth in SEQ ID NO: 1, wherein X1 is N, X2 is selected from any amino acid of D and Y, X3 is D, X4 is D, X5 is E, X6 is I, X7 is N, X8 is Q, X9 is M, and X10 is I.

13. The IL-15 mutant polypeptide according to claim 1, wherein the IL-15 mutant polypeptide comprises the amino acid sequence as set forth in SEQ ID NO: 1, wherein X1 is N, X2 is S, X3 is F, X4 is D, X5 is E, X6 is I, X7 is N, X8 is Q, X9 is M, and X10 is I.

14. The IL-15 mutant polypeptide according to claim 1, wherein the IL-15 mutant polypeptide comprises the amino acid sequence as set forth in SEQ ID NO: 1, wherein X1 is N, X2 is S, X3 is D, X4 is selected from any amino acid of G and N, X5 is E, X6 is I, X7 is N, X8 is Q, X9 is M, and X10 is I.

15. The IL-15 mutant polypeptide according to claim 1, wherein the IL-15 mutant polypeptide comprises the amino acid sequence as set forth in SEQ ID NO: 1, wherein X1 is N, X2 is S, X3 is D, X4 is D, X5 is Y, X6 is I, X7 is N, X8 is Q, X9 is M, and X10 is I.

16. The IL-15 mutant polypeptide according to claim 1, wherein the IL-15 mutant polypeptide comprises the amino acid sequence as set forth in SEQ ID NO: 1, wherein X1 is N, X2 is S, X3 is D, X4 is D, X5 is E, X6 is I, X7 is Y, X8 is Q, X9 is M, and X10 is I.

17. The IL-15 mutant polypeptide according to claim 1, wherein the IL-15 mutant polypeptide comprises the amino acid sequence as set forth in SEQ ID NO: 1, wherein X1 is N, X2 is S, X3 is D, X4 is D, X5 is E, X6 is I, X7 is Y, X8 is selected from any amino acid of S, F and A, X9 is M, and X10 is I.

18. The IL-15 mutant polypeptide according to claim 1, wherein the IL-15 mutant polypeptide comprises the amino acid sequence as set forth in SEQ ID NO: 1, wherein X1 is N, X2 is S, X3 is D, X4 is D, X5 is E, X6 is I, X7 is N, X8 is Q, X9 is H, and X10 is I.

19. The IL-15 mutant polypeptide according to claim 1, wherein the IL-15 mutant polypeptide comprises the amino acid sequence as set forth in SEQ ID NO: 1, wherein X1 is N, X2 is S, X3 is D, X4 is D, X5 is E, X6 is I, X7 is N, X8 is Q, X9 is M, and X10 is selected from any amino acid of A and H.

20. A method for treating an autoimmune disease, an inflammatory disease, a neurodegenerative disease, a cancer or a pathogen infection, comprising administering to a subject a therapeutically effective amount of the nucleic acid molecule according to claim 4.


SEQUENCE LISTING (17 sequences)

SEQ ID NO: 1 - protein, 114 aa, Artificial Sequence
  interleukin-15 mutant
  misc_feature (4)..(4): Xaa can be Ala or Asn
  misc_feature (7)..(7): Xaa can be Ser, Asp, or Tyr
  misc_feature (8)..(8): Xaa can be Asp or Phe
  misc_feature (61)..(61): Xaa can be Asp, Gly, or Asn
  misc_feature (64)..(64): Xaa can be Glu or Tyr
  misc_feature (68)..(68): Xaa can be Ile, Gln, or Lys
  misc_feature (72)..(72): Xaa can be Asn or Tyr
  misc_feature (108)..(108): Xaa can be Gln, Ser, Phe, or Ala
  misc_feature (109)..(109): Xaa can be Met or His
  misc_feature (111)..(111): Xaa can be Ile, Ala, or His
NWVXVIXXLKKIEDLIQSMHIDATLYTESDVHPSCKVTAMKCFLLELQVISLESGDASIHXTVXNLIXLANXSLSSNGNVTESGCKECEELEEKNIKEFLQSFVHIVXXFXNTS

SEQ ID NO: 2 - protein, 114 aa, Artificial Sequence
  interleukin-15 mutant
NWVAVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 114 aa, Artificial Sequence
  interleukin-15 mutant
NWVNVIDDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 4 - protein, 114 aa, Artificial Sequence
  interleukin-15 mutant
NWVNVIYDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 5 - protein, 114 aa, Artificial Sequence
  interleukin-15 mutant
NWVNVISFLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 6 - protein, 114 aa, Artificial Sequence
  interleukin-15 mutant
NWVNVISDLKKIEDLIQSMHIDATLYTESDVHPSCKVTAMKCFLLELQVISLESGDASIHGTVENLIILANNSLSSNGNVTESGCKECEELEEKNIKEFLQSFVHIVQMFINTS

SEQ ID NO: 7 - protein, 114 aa, Artificial Sequence
  interleukin-15 mutant
NWVNVISDLKKIEDLIQSMHIDATLYTESDVHPSCKVTAMKCFLLELQVISLESGDASIHNTVENLIILANNSLSSNGNVTESGCKECEELEEKNIKEFLQSFVHIVQMFINTS

SEQ ID NO: 8 - protein, 114 aa, Artificial Sequence
  interleukin-15 mutant
NWVNVISDLKKIEDLIQSMHIDATLYTESDVHPSCKVTAMKCFLLELQVISLESGDASIHDTVYNLIILANNSLSSNGNVTESGCKECEELEEKNIKEFLQSFVHIVQMFINTS

SEQ ID NO: 9 - protein, 114 aa, Artificial Sequence
  interleukin-15 mutant
NWVNVISDLKKIEDLIQSMHIDATLYTESDVHPSCKVTAMKCFLLELQVISLESGDASIHDTVENLIQLANNSLSSNGNVTESGCKECEELEEKNIKEFLQSFVHIVQMFINTS

SEQ ID NO: 10 - protein, 114 aa, Artificial Sequence
  interleukin-15 mutant
NWVNVISDLKKIEDLIQSMHIDATLYTESDVHPSCKVTAMKCFLLELQVISLESGDASIHDTVENLIKLANNSLSSNGNVTESGCKECEELEEKNIKEFLQSFVHIVQMFINTS

SEQ ID NO: 11 - protein, 114 aa, Artificial Sequence
  interleukin-15 mutant
NWVNVISDLKKIEDLIQSMHIDATLYTESDVHPSCKVTAMKCFLLELQVISLESGDASIHDTVENLIILANYSLSSNGNVTESGCKECEELEEKNIKEFLQSFVHIVQMFINTS

SEQ ID NO: 12 - protein, 114 aa, Artificial Sequence
  interleukin-15 mutant
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVSMFINTS

SEQ ID NO: 13 - protein, 114 aa, Artificial Sequence
  interleukin-15 mutant
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVFMFINTS

SEQ ID NO: 14 - protein, 114 aa, Artificial Sequence
  interleukin-15 mutant
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVAMFINTS

SEQ ID NO: 15 - protein, 114 aa, Artificial Sequence
  interleukin-15 mutant
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQHFINTS

SEQ ID NO: 16 - protein, 114 aa, Artificial Sequence
  interleukin-15 mutant
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFANTS

SEQ ID NO: 17 - protein, 114 aa, Artificial Sequence
  interleukin-15 mutant
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFHNTS